(19) United States
(12) Patent Application Publication
(10) Pub. No.: US 2023/0218213 A1
(43) Pub. Date: Jul. 13, 2023

(54) FMRI-HIPPOCAMPUS ACOUSTIC BATTERY (FHAB)

(71) Applicant: The Johns Hopkins University, Baltimore, MD (US)

(72) Inventor: Una D. McCann, Baltimore, MD (US)

(21) Appl. No.: 18/011,068

(22) PCT Filed: Jul. 1, 2021
(86) PCT No.: PCT/US2021/040100

§ 371 Date: Dec. 16, 2022

Related U.S. Application Data
(60) Provisional application No. 63/047,541, filed on Jul. 2, 2020.

(51) Int. Cl. A61B 5/16; A61B 5/00; A61M 21/00

(52) U.S. Cl. CPC A61B 5/165; A61B 5/4064; A61M 21/00; A61M 2021/0027

(57) ABSTRACT

The present disclosure relates to materials and methods for evaluating acoustic startle response (ASR) and pre-pulse inhibition (PPI) in a subject. In particular, the present disclosure relates to a set of acoustic signals and their use in methods for evaluation and/or treatment of mental disorder in a subject. The methods comprise delivering a set of acoustic signals as described herein to the subject, and measuring the startle response in the subject. The startle response may be the blink reflex, pupil dilation, skin conductive response, and/or brain activity in fMRI. For example, measuring the blink reflex may involve measuring the speed, magnitude, and/or duration of the blink reflex in the subject.

### FIELD

The present disclosure relates to materials and methods for evaluating acoustic startle response (ASR) and pre-pulse inhibition (PPI) in a subject. In particular, the present disclosure relates to a set of acoustic signals and their use in methods for evaluation and/or treatment of mental disorder in a subject.

### BACKGROUND

Trauma exposure is a common occurrence, and depending on the severity, repetition of events, and recovery, may lead to various psychiatric disorders in some individuals. According to the National Institute of health, nearly one in five adults live with a mental disorder, such as depression, anxiety, bipolar disorders, personality disorders, psychotic disorders, and trauma related disorders, especially posttraumatic stress disorder (PTSD) [1]. Many of these conditions have overlapping symptoms within the body's autonomic nervous system (fight and flight/rest and digest), such as hypervigilance, sleep disturbances, recurring and invasive thoughts, difficulty concentrating, and anomalous cardio rhythms [2]. The comorbidity of shared symptoms within these disorders (and potentially others) make diagnosis of a specific disorder costly and difficult. Accordingly, improved methods for diagnosis and subsequent treatment of specific disorders, such as PTSD, are needed.

### SUMMARY

In some aspects, provided herein is a set of acoustic signals for delivery to a subject. The set comprises at least one pre-pulse stimulus and at least one pulse stimulus. In some embodiments, the at least one pre-pulse stimulus has a frequency of 200 Hz, 300 Hz, 400 Hz, 500 Hz, 1000 Hz, 3000 Hz, 4000 Hz, or 5000 Hz. In some embodiments, the pre-pulse stimulus has an amplitude of 15 dB, 25 dB, or 45 dB. In some embodiments, the at least one pre-pulse stimulus has a frequency of 3000 Hz. In some embodiments, the pulse stimulus has a frequency of 200 Hz, 300 Hz, 400 Hz, 500 Hz, 1000 Hz, 3000 Hz, 4000 Hz, or 5000 Hz and an amplitude of 65 dB, 80 dB, or 105 dB. In some embodiments, the at least one pulse stimulus has a frequency of 3000 Hz.

In some aspects, provided herein are methods for evaluating an acoustic startle reflex in a subject. The methods comprise delivering a set of acoustic signals as described herein to the subject, and measuring the startle response in the subject. The startle response may be the blink reflex, pupil dilation, skin conductive response, and/or brain activity in fMRI. For example, measuring the blink reflex may involve measuring the speed, magnitude, and/or duration of the blink reflex in the subject. In some embodiments, an increased startle response in the subject indicates that the subject has a mental disorder. In some embodiments, measuring the startle response may comprise measuring brain activity in the hippocampus by fMRI. Increased activity in the hippocampus as measured by fMRI may indicate that the subject has post-traumatic stress disorder (PTSD).

In some aspects, provided herein are methods for performing acoustic neuromodulation in a subject. The methods comprise (a) delivering an acoustic stimulus to the subject, (b) measuring an initial startle response in the subject, (c) delivering the acoustic stimulus to the subject, (d) measuring a subsequent startle response in the subject, and (e) comparing the startle responses in the subject. In some embodiments, the methods further comprise and repeating steps (a)-(d) when the subsequent startle response in the subject is not reduced compared to the initial startle response. In some embodiments, the methods comprise ceasing delivery of the acoustic stimulus to the subject when the subsequent startle response in the subject is reduced compared to the initial startle response.

The acoustic stimulus may comprise a pulse stimulus as described herein. For example, the acoustic stimulus may comprise a pulse stimulus having a frequency of 200 Hz, 300 Hz, 400 Hz, 500 Hz, 1000 Hz, 3000 Hz, 4000 Hz, or 5000 Hz and an amplitude of 65 dB, 80 dB, or 105 dB. In some embodiments, the methods further comprise delivering a pre-pulse stimulus to the subject prior the pulse stimulus. In some embodiments, the pre-pulse stimulus is delivered to the subject prior to each pulse stimulus. The pre-pulse stimulus may have a frequency of 200 Hz, 300 Hz, 400 Hz, 500 Hz, 1000 Hz, 3000 Hz, 4000 Hz, or 5000 Hz and an amplitude of 15 dB, 25 dB, or 45 dB.

In some embodiments, measuring the initial and subsequent startle response in the subject comprises obtaining the same measurement from the subject. The measurement may be selected from the blink reflex, pupil dilation, skin conductive response, and brain activity in fMRI. Measuring the blink reflex may comprise measuring the speed, magnitude, and/or duration of the blink reflex in the subject. Measuring brain activity in fMRI may comprise measuring activity in the hippocampus.

In some embodiments, provided herein are methods for evaluating paired-pulse inhibition (PPI) in a subject. The method may comprise performing the following steps in order:
a. Delivering a pulse stimulus to the subject;
b. Measuring a first startle response in the subject;
c. Delivering a pre-pulse stimulus to the subject;
d. Delivering the pulse stimulus to the subject;
e. Measuring a second startle response in the subject; and
f. Comparing the second startle response to the first startle response in the subject.

In some embodiments, measuring the first and second startle response comprises obtaining the same measurement from the subject. The measurement may be selected from the blink reflex, pupil dilation, skin conductive response, and brain activity in fMRI. In some embodiments, the pre-pulse stimulus has a frequency of 200 Hz, 300 Hz, 400 Hz, 500 Hz, 1000 Hz, 3000 Hz, 4000 Hz, or 5000 Hz and an amplitude of 15 dB, 25 dB, or 45 dB. In some embodiments, the pulse stimulus has a frequency of 200 Hz, 300 Hz, 400 Hz, 500 Hz, 1000 Hz, 3000 Hz, 4000 Hz, or 5000 Hz and an amplitude of 65 dB, 80 dB, or 105 dB.

Other aspects and embodiments of the disclosure will be apparent in light of the following detailed description.

### DETAILED DESCRIPTION OF THE INVENTION

### 1. Definitions

To facilitate an understanding of the present technology, a number of terms and phrases are defined below. Additional definitions are set forth throughout the detailed description.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The terms “acoustic startle reflex” or “ASR” as used interchangeably herein refer to muscular activity produced reflexively in response to an auditory stimulus (e.g., sound).

The term “amplitude” as used herein with relation to an acoustic signal refers to the intensity of the signal. In some embodiments, the intensity of the signal is described in decibels. Signals of a higher amplitude (e.g., higher intensity) are perceived as louder than signals of a lower amplitude (e.g., lower intensity).

The term “frequency” as used herein with relation to an acoustic signal refers to the number of waves (e.g., sound waves) that pass a fixed point in a given amount of time. In some embodiments, the “frequency” of an acoustic signal is described in Hertz (e.g., units per second). Auditory signals of a higher frequency are perceived as higher in pitch than auditory signals of a lower frequency.

The term “functional magnetic resonance imaging” or “fMRI” as used interchangeably herein refers to a non-invasive technique for measuring brain and/or spinal cord activity by detecting changes associated with blood flow and/or oxygenation in a given area. fMRI is grounded in the fact that blood flow and neuronal activation are coupled, such that when an area of the brain or spinal cord is “active”, blood flow to the region increases. fMRI may use the blood-oxygen-level dependent (BOLD) contrast, which maps neural activity in the brain or spinal cord by imaging the hemodynamic response related to energy use in a given area. In some embodiments, fMRI may be performed during a task or stimulus. In other embodiments, fMRI can be used to measure a subjects resting state, or baseline activity.

As used herein, the term “mental disorder” is used in the broadest sense and refers to a wide range of conditions that affect mood, thinking, and/or behavior. In some embodiments, a mental disorder refers to a condition associated with a hyperactive acoustic startle reflex in a subject. In some embodiments, a mental disorder refers to a condition associated with diminished paired pulse inhibition in a subject. Mental disorders include, for example, depression, anxiety disorders (e.g., panic disorder, obsessive-compulsive disorder, phobias), bipolar disorders, personality disorders (e.g., borderline personality disorder, narcissistic personality disorder, paranoia and delusional disorders, schizoid personality disorder, schizotypal personality disorder) psychotic disorders (e.g., schizophrenia, psychosis), and trauma-related disorders (e.g., post-traumatic stress disorder, acute stress disorder, adjustment disorders, reactive attachment disorder, disinhibited social engagement disorder).

As used herein, the term “neuromodulation” refers to alteration of cellular (e.g., neuronal) activity through targeted delivery of a stimulus. The stimulus may be an acoustic stimulus. Neuromodulation using an acoustic stimulus is referred to herein as “acoustic neromodulation.”

The term “orbicularis oculi” as used herein refers to the muscle in the face that closes the eyelids.

The terms “pre-pulse inhibition” or “PPI” as used interchangeably herein refer to a neurological phenomenon in which a weak pre-stimulus (e.g., pre-pulse) inhibits the reaction of an organism to a subsequent, stronger stimulus (pulse). In the context of the present disclosure, the terms “pre-pulse” and “pulse” each refer to auditory signals (e.g., sounds). For example, PPI may refer to a phenomenon in which an auditory pre-pulse inhibits the ASR in response to a second auditory pulse.

As used herein, the term “startle response” refers to any suitable measurement of the startle response in a subject. Suitable startle responses include, for example, the blink reflex (e.g., movement of the orbicularis oculi of the eye), skin conductance, pupil dilation, and fMRI.

As used herein, the terms “treat,” “treatment,” and “treating” refer to reducing the amount or severity of a particular condition, disease state (e.g., a mental disorder), or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

### 2. Signals, Methods, and Devices

Measurement of the acoustic startle reflex (ASR) and/or pre-pulse inhibition (PPI) thereof may serve as a viable means for diagnosing a variety of mental disorders. Optimal pre-pulse and pulse stimuli for most effective measurement of the ASR or PPI in a subject, however, are unknown. Moreover, the diagnosis of a specific mental disorder (e.g., PTSD) may rely on analysis of specific brain areas that are activated (or not activated) during ASR induction and/or PPI.

### a. Set of Acoustic Signals

In some aspects, provided herein is a set of acoustic signals. The set of acoustic signals comprises stimuli (e.g., sounds) of varying amplitudes and frequencies that may be delivered to a subject. In some embodiments, the set of acoustic signals comprises at least one pre-pulse stimulus and at least one pulse stimulus. In general, pre-pulse stimuli comprise acoustic signals of lower amplitudes (e.g., lower intensities) compared to pulse stimuli. Pre-pulse stimuli may comprise acoustic signals of amplitudes ranging from 10-100 dB. For example, pre-pulse stimuli may comprise acoustic signals having amplitudes of 10 dB, 15 dB, 20 dB, 25 dB, 30 dB, 35 dB, 40 dB, 45 dB, 50 dB, 55 dB, 60 dB, 65 dB, 70 dB, 75 Db, 80 dB, 85 Db, 90 dB, 95 dB, or 100 dB. Any suitable pre-pulse stimuli may be used, provided that the pre-pulse stimulus is less intense (e.g. of a lower amplitude) than the pulse stimulus.

In some embodiments, the amplitude of the pre-pulse stimulus is 5 dB lower than the amplitude of the pulse stimulus. For example, the amplitude of the pre-pulse stimulus may be 100 dB and the amplitude of the pulse stimulus may be 105 dB. Alternatively, the amplitude of the pre-pulse stimulus may be 75 dB and the amplitude of the pulse stimulus may be 80 dB. As another example, the amplitude of the pre-pulse stimulus may be 60 dB and the amplitude of the pulse stimulus may be 65 dB.

In some embodiments, the amplitude of the pre-pulse stimulus is more than 5 dB (e.g. 10 dB, 15 dB, 20 dB, 25 dB, 30 dB, 35 dB, 40 dB, 45 dB, 50 dB, 55 dB, 60 dB, 65 dB, 70 dB, 75 dB, 80 dB, 85 dB, or 90 dB) lower than the pulse stimulus. For example, the amplitude of the pre-pulse stimulus may be 15 dB and the amplitude of the pulse stimulus may be 65 dB-105 dB (e.g. 65 dB, 80 dB, 105 dB). As another example, the amplitude of the pre-pulse stimulus may be 25 dB and the amplitude of the pulse stimulus may be 65 dB-105 dB (e.g. 65 dB, 80 dB, 105 dB).

In some embodiments, the frequency of pre-pulse stimuli may be 200 Hz to 5000 Hz. For example, the frequency may be 200 Hz, 300 Hz, 400 Hz, 500 Hz, 1000 Hz, 3000 Hz, 4000 Hz, or 5000 Hz. Any suitable combination of frequency and amplitude for the pre-pulse stimulus may be used. In some embodiments, the pre-pulse stimulus has an amplitude of 10 dB-100 dB and a frequency of 200-5000 Hz. For example, a pre-pulse stimulus may have an amplitude of 15 dB and a frequency of 200 Hz, 300 Hz, 400 Hz, 500 Hz, 1000 Hz, 3000 Hz, 4000 Hz, or 5000 Hz. As another example, a pre-pulse stimulus may have an amplitude of 25 dB and a frequency of 200 Hz, 300 Hz, 400 Hz, 500 Hz, 1000 Hz, 3000 Hz, 4000 Hz, or 5000 Hz. As another example, a pre-pulse stimulus may have an amplitude of 45 dB and a frequency of 200 Hz, 300 Hz, 400 Hz, 500 Hz, 1000 Hz, 3000 Hz, 4000 Hz, or 5000 Hz. In particular embodiments, the frequency of the pre-pulse stimulus is 3000 Hz. For example, the pre-pulse stimulus may have a frequency of 3000 Hz and an amplitude of 15 dB.

Pulse stimuli may comprise acoustic stimuli of amplitudes ranging from 65 dB to 105 dB. For example, pulse stimuli may comprise acoustic signals having amplitudes of 65 dB, 80 dB, or 105 dB. The frequency of pulse stimuli may be 200 Hz to 5000 Hz. For example, the frequency may be 200 Hz, 300 Hz, 400 Hz, 500 Hz, 1000 Hz, 3000 Hz, 4000 Hz, or 5000 Hz. Any suitable combination of frequency and amplitude for the pulse stimulus may be used. For example, the pulse stimulus may have an amplitude of 65-105 dB and a frequency of 200-5000 Hz. For example, a pulse stimulus may have an amplitude of 65 dB and a frequency of 200 Hz, 300 Hz, 400 Hz, 500 Hz, 1000 Hz, 3000 Hz, 4000 Hz, or 5000 Hz. As another example, a pulse stimulus may have an amplitude of 80 dB and a frequency of 200 Hz, 300 Hz, 400 Hz, 500 Hz, 1000 Hz, 3000 Hz, 4000 Hz, or 5000 Hz. As another example, a pulse stimulus may have an amplitude of 105 dB and a frequency of 200 Hz, 300 Hz, 400 Hz, 500 Hz, 1000 Hz, 3000 Hz, 4000 Hz, or 5000 Hz. In particular embodiments, the frequency of the pulse stimulus is 3000 Hz. For example, the pulse stimulus may have a frequency of 3000 Hz and an amplitude of 105 dB.

Exemplary amplitudes and frequencies of pre-pulse and pulse stimuli are shown in Table 1 below.

TABLE 1
Exemplary Amplitudes and Frequencies of Acoustic Stimuli

| | | | | | | | |
|---|---|---|---|---|---|---|---|
| 15 dB_200 | 15 dB_300 | 15 dB_400 | 15 dB_500 | 15 dB_1000 | 15 dB_3000 | 15 dB_4000 | 15 dB_5000 |
| Hz | Hz | Hz | Hz | Hz | Hz | Hz | Hz |
| 25 dB_200 | 25 dB_300 | 25 dB_400 | 25 dB_500 | 25 dB_1000 | 25 dB_3000 | 25 dB_4000 | 25 dB_5000 |
| Hz | Hz | Hz | Hz | Hz | Hz | Hz | Hz |
| 45 dB_200 | 45 dB_300 | 45 dB_400 | 45 dB_500 | 45 dB_1000 | 45 dB_3000 | 45 dB_4000 | 45 dB_5000 |
| Hz | Hz | Hz | Hz | Hz | Hz | Hz | Hz |
| 65 dB_200 | 45 dB_300 | 45 dB_400 | 45 dB_500 | 45 dB_1000 | 45 dB_3000 | 45 dB_4000 | 45 dB_5000 |
| Hz | Hz | Hz | Hz | Hz | Hz | Hz | Hz |
| 80 dB_200 | 80 dB_300 | 80 dB_400 | 80 dB_500 | 80 dB_1000 | 80 dB_3000 | 80 dB_4000 | 80 dB_5000 |
| Hz | Hz | Hz | Hz | Hz | Hz | Hz | Hz |
| 105 dB_200 | 105 dB_300 | 105 dB_400 | 105 dB_500 | 105 dB_1000 | 105 dB_3000 | 105 dB_4000 | 105 dB_5000 |
| Hz | Hz | Hz | Hz | Hz | Hz | Hz | Hz |

In some embodiments, the set of acoustic signals comprises one pre-pulse stimulus. In some embodiments, the set of acoustic signals comprises multiple pre-pulse stimuli. For example, 2, 3, 4, 5, 6, 7, 8, 9, 10, or more than 10 pre-pulse stimuli may be delivered to a subject as a pre-stimulus for evaluation of PPI. In particular embodiments, 6 pre-pulse stimuli may be delivered to the subject prior to a single, higher intensity pulse signal. For example, 6 pre-pulse stimuli having an amplitude of 15 dB and a frequency of 3000 Hz may be delivered to the subject prior to the pulse stimulus. In some embodiments, the pre-pulse stimuli each have the same amplitude. For example, the pre-pulse stimuli may each have an amplitude of 15 dB. In some embodiments, one or more pre-pulse stimuli is of a different amplitude than at least one other pre-pulse stimulus. For example, at least one pre-pulse stimulus may have an amplitude of 15 dB and at least one pre-pulse stimulus may have an amplitude of 25 dB.

The pre-pulse stimulus or the pre-pulse stimuli may be delivered to a subject for any suitable duration prior to the pulse stimulus. For example, the pre-pulse stimulus may be delivered for 1 ms-500 ms. For example, the pre-pulse stimulus may be delivered for 1 ms, 2 ms, 3 ms, 4 ms, 5 ms, 6 ms, 7 ms, 8 ms, 9 ms, 10 ms, 20 ms, 30 ms, 40 ms, 50 ms, 60 ms, 70 ms, 80 ms, 90 ms, 100 ms, 120 ms, 140 ms, 160 ms, 180 ms, 200 ms, 220 ms, 240 ms, 260 ms, 280 ms, 300 ms, 320 ms, 340 ms, 360 ms, 380 ms, 400 ms, 420 ms, 440 ms, 460 ms, 480, or 500 ms. In some embodiments, multiple pre-pulse stimuli are delivered. In such embodiments, the duration of each pre-pulse stimuli may be any suitable duration.

In embodiments, where multiple pre-pulse stimuli are delivered to a subject, the pre-pulse stimuli may be spaced at any suitable interval. For example, the pre-pulse stimuli may be spaced 1 ms-10 seconds apart. For example, the pre-pulse stimuli may be spaced 1 ms, 10 ms, 20 ms, 30 ms, 40 ms, 50 ms, 60 ms, 70 ms, 80 ms, 90 ms, 100 ms, 150 ms, 200 ms, 250 ms, 300 ms, 350 ms, 400 ms, 450 ms, 500 ms, 550 ms, 600 ms, 650 ms, 700 ms, 750 ms, 800 ms, 850 ms, 900 ms, 950 ms, 1 second, 2 seconds, 3 seconds, 4 seconds, 5 seconds, 6 seconds, 7 seconds, 8 seconds, 9 seconds, or 10 seconds apart.

The pre-pulse stimulus, or the last of the pre-pulse stimuli, may be spaced from the pulse stimulus by any suitable interval. In some embodiments, the pre-pulse stimulus or the last of the pre-pulse stimuli is provided to the subject no more than 500 ms prior to the pulse stimulus. For example, the pre-pulse stimulus or the last of the pre-pulse stimuli may be provided to the subject between 1 ms-10 s prior to the pulse stimulus. For example, the pre-pulse stimulus or the last of the pre-pulse stimuli may be provided to the subject 1 ms, 5 ms, 10 ms, 15 ms, 20 ms, 25 ms, 30 ms, 35 ms, 40 ms, 45 ms, 50 ms, 55 ms, 60 ms, 65 ms, 70 ms, 75 ms, 80 ms, 85 ms, 90 ms, 95 ms, 100 ms, 110 ms, 120 ms, 130 ms, 140 ms, 150 ms, 160 ms, 170 ms, 180 ms, 190 ms, 200 ms, 210 ms, 220 ms, 230 ms, 240 ms, 250 ms, 260 ms, 270 ms, 280 ms, 290 ms, 300 ms, 310 ms, 320 ms, 330 ms, 340 ms, 350 ms, 360 ms, 370 ms, 380 ms, 390 ms, 400 ms, 410 ms, 420 ms, 430 ms, 440 ms, 450 ms, 460 ms, 470 ms, 480 ms, 490 ms, or 500 ms prior to the pulse stimulus. In some embodiments, the pre-pulse stimulus or the last of the pre-pulse stimuli may be provided to the subject more than 500 ms (e.g., 550 ms, 600 ms, 650 ms, 700 ms, 750 ms, 800 ms, 850 ms, 900 ms, 950 ms, 1 second, 2 seconds, 3 seconds, 4 seconds, 5 seconds, 6 seconds, 7 seconds, 8 seconds, 9 seconds, or 10 seconds) prior to the pulse stimulus.

### b. Methods for Evaluating ASR

In some embodiments, provided herein are methods for evaluating the ASR in a subject. The methods may comprise delivering to the subject a set of acoustic signals comprising at least one pre-pulse stimulus and at least one pulse stimulus as described herein. The methods may further comprise measuring one or more startle responses in the subject. For any startle responses measured, baseline measurements in the subject prior to exposure to the pre-pulse stimulus may be established to ensure accuracy of the startle response measurement following exposure to the stimulus. In some embodiments, a single type of startle response in the subject is measured to evaluate the ASR in the subject. In some embodiments, multiple types of startle responses may be measured.

In some embodiments, startle responses comprise unconscious movements in the orbicularis oculi of the eye. Such unconscious movements are referred to herein as a “blink reflex”. For example, muscle movements of the orbicularis oculi may be measured by electromyogram (EMG), such as by EMG using electro-oculo-graphic (EOG) electrodes. Alternatively, muscle movements of the orbicularis oculi may be measured by a camera. For example, a suitable camera which captures the speed, magnitude, and/or duration of the blink reflex may be used to measure ASR in a subject. Suitable cameras and methods of use thereof are provided in U.S. Patent Publication No. 20190008435, the entire contents of which are incorporated herein by reference. An abnormal ASR may be characterized by increased speed, magnitude, and/or duration of the blink reflex in response to the pulse stimulus compared to a control. For example, measurements of the speed, magnitude, and/or duration of the blink reflex in response to a pulse stimulus may be obtained for a healthy control subject or a group of healthy control subjects. The blink reflex measurements of a subject may be compared to these control values to determine whether the subject has an abnormal ASR.

In some embodiments, a type of startle response that may be measured is brain activity in fMRI. For example, the subject may be undergoing fMRI before, during, and/or after exposure to the acoustic stimuli (e.g., pre-pulse and pulse stimuli) described herein. Accordingly, activity in a desired brain area may be measured before, during and/or after exposure to the acoustic stimuli. For example, the resting state fMRI data may be collected in the subject to determine baseline activity in one or more areas of the brain. Following determination of baseline activity, the subject may be exposed to a pre-pulse stimulus and subsequently exposed to a pulse stimulus. fMRI data may be collected and used to assess brain activity during and/or after delivery of the stimuli to the subject. In particular embodiments, fMRI data may be collected to evaluate the ASR in the subject. For example, fMRI data may be collected to evaluate whether the subject has a normal or an abnormal ASR. An abnormal ASR may be characterized by a hyperactive response to the pre-pulse and/or pulse stimulus compared to a control. For example, fMRI data may be obtained for a healthy control subject or a group of healthy control subjects, and the fMRI data from a subject may be compared to these control values to determine whether the subject has a hyperactive response to the pre-pulse and/or pulse stimulus.

Other suitable types of startle responses that may be measured include pupil dilation and/or the skin conductive response. Pupil dilation may be measured by any suitable means, such as a camera. Skin conductance may be measured by electrodes, such as electrodes placed on the fingers or the hand. As described for the blink reflex and fMRI methods, pupil dilation and/or skin conductance measurements may be compared to control values to determine whether a subject has a hyperactive ASR.

### c. Methods for Evaluating PPI

In some embodiments, provided herein are methods for evaluating PPI in a subject. The methods may comprise delivering to the subject a set of acoustic signals comprising at least one pre-pulse stimulus and at least one pulse stimulus as described herein and measuring a startle response in the subject. For any startle responses measured, baseline measurements in the subject prior to exposure to the pre-pulse stimulus may be established to ensure accuracy of the startle response following exposure to the stimulus. In some embodiments, a single type of startle response in the subject is measured to evaluate PPI in the subject. In some embodiments, multiple types of startle responses may be measured.

In some embodiments, the method comprises measuring a startle responses in the subject in response to the pulse stimulus, and determining whether the startle response is diminished following a previous exposure to the pre-pulse stimulus compared to response to the pulse stimulus alone. For example, a subject may be exposed to a pulse stimulus and a first startle response in the subject may be measured. In a subsequent trial, the subject may be exposed to at least one pre-pulse stimulus and subsequently exposed to the pulse stimulus, and a second startle response in the subject may be measured. The subject may be exposed to any suitable number of rounds of stimulus delivery (e.g., pre-pulse and pulse stimuli). For example, in a third trial, the subject may be exposed to at least one pre-pulse stimulus and subsequently exposed to the pulse stimulus, and a third startle response in the subject may be measured. A subject may be exposed to a fourth trial, a fifth trial, a sixth trial, and so forth. A subject exhibiting PPI will demonstrate a reduced response to the pulse stimulus following a prior exposure to the pre-pulse stimulus. In other words, the second startle response (or the third startle response, the fourth startle response, etc.) in the subject will be reduced compared to the first startle response. In contrast, a subject exhibiting abnormal PPI will not demonstrate such a reduction in response to the pulse stimulus, even following prior exposure to the pre-pulse stimulus.

In some embodiments, a startle response comprise unconscious movements in the orbicularis oculi of the eye. Such unconscious movements are referred to herein as a “blink reflex”. For example, muscle movements of the orbicularis oculi may be measured by electromyogram (EMG), such as by EMG using electro-oculo-graphic (EOG) electrodes. Alternatively, muscle movements of the orbicularis oculi may be measured by a camera. For example, a suitable camera which captures the speed, magnitude, and/or duration of the blink reflex may be used to measure PPI in a subject. An abnormal PPI may be characterized by a similar speed, magnitude, and/or duration of the blink reflex in trials where the pulse stimulus is preceded by a pre-pulse stimulus and trials where the pulse stimulus is delivered alone.

In some embodiments, a type of startle response that may be measured is brain activity in fMRI. For example, the subject may be undergoing fMRI before, during, and/or after exposure to the acoustic stimuli (e.g., pre-pulse and pulse stimuli) described herein. Accordingly, activity in a desired brain area may be measured before, during and/or after exposure to the acoustic stimuli. For example, the resting state fMRI data may be collected in the subject to determine baseline activity in one or more areas of the brain. Following determination of baseline activity, the subject may be exposed to a pulse stimulus. In some embodiments, fMRI data may be collected to evaluate whether the subject experiences PPI, wherein exposure to the at least one pre-pulse stimulus results in diminished activity (e.g., BOLD activity) in an area of the brain following the pulse stimulus compared to activity following the pulse stimulus alone. For example, fMRI data may be collected and used to assess brain activity during and/or after delivery of the pulse stimulus to the subject. In a following trial, the subject may be exposed to a pre-pulse stimulus and subsequently exposed to a pulse stimulus. fMRI data may be collected and used to assess whether brain activity during and/or after delivery of the pulse stimulus is reduced following exposure to the pre-pulse stimulus compared to the trial where the pulse stimulus was delivered without a preceding pre-pulse stimulus.

Other suitable types of startle responses that may be measured include pupil dilation and/or the skin conductive response. Pupil dilation may be measured by any suitable means, such as a camera. Skin conductance may be measured by electrodes, such as electrodes placed on the fingers or the hand. As described for the blink reflex and fMRI methods, pupil dilation and/or skin conductance measurements may be measured following delivery of a pulse stimulus in an initial trial, and compared to a subsequent trial where the pulse stimulus is preceded by a pre-pulse stimulus.

### d. Evaluating Mental Disorders

In some embodiments, the methods for evaluating the ASR and/or PPI in a subject may be used to evaluate whether the subject has a mental disorder. Mental disorders include, for example, depression, anxiety disorders (e.g., panic disorder, obsessive-compulsive disorder, phobias), bipolar disorders, personality disorders (e.g., borderline personality disorder, narcissistic personality disorder, paranoia and delusional disorders, schizoid personality disorder, schizotypal personality disorder) psychotic disorders (e.g., schizophrenia, psychosis), and trauma-related disorders (e.g., post-traumatic stress disorder, acute stress disorder, adjustment disorders, reactive attachment disorder, disinhibited social engagement disorder). For example, a subject with an abnormal (e.g., hyperactive) ASR may be diagnosed with a mental disorder. As another example, a subject with abnormal (e.g., lack of, diminished) PPI may be diagnosed with a mental disorder. In some embodiments, methods for evaluating the ASR and/or PPI in a subject may be used to evaluate whether the subject is suffering from post-traumatic stress disorder (PTSD).

In some embodiments, a subject may be determined to have an abnormal ASR and/or PPI based upon fMRI measurements. For example, fMRI may be used to evaluate activity in any suitable area of the brain before, during, and/or after exposure to the acoustic stimuli described herein. Suitable areas include, for example, the hippocampus, the amygdala, the hypothalamus, and/or the prefrontal cortex. In some embodiments, fMRI may be used to evaluate activity in the hippocampus. In some embodiments, activity in the hippocampus may be evaluated to assist with the diagnosis of a specific mental disorder. For example, activity in the hippocampus may be evaluated to assist with the diagnosis of PTSD in a subject. For example, abnormal activity in the hippocampus (e.g., hyperactivity, as measured by enhanced BOLD contrast compared to control) following exposure to an acoustic signal may be indicative of PTSD in a subject.

### e. Acoustic Neuromodulation

In some embodiments, provided herein are methods for performing acoustic neuromodulation in a subject. In some embodiments, the methods comprise delivering to the subject at least one pre-pulse stimulus and/or at least one pulse stimulus as described herein. In some embodiments, the methods comprise delivering to the subject a set of acoustic signals as described herein.

In some embodiments, methods for performing acoustic neuromodulation in a subject comprise delivering an acoustic stimulus to the subject and measuring an initial startle response in the subject. The acoustic stimulus may be a pulse stimulus as described herein. For example, the acoustic stimulus may be a pulse stimulus having a frequency of 200 Hz-5000 Hz and an amplitude of 65 dB-105 dB. For example, the pulse stimulus may have a frequency of 200 Hz, 300 Hz, 400 Hz, 500 Hz, 1000 Hz, 3000 Hz, 4000 Hz, or 5000 Hz and an amplitude of 65 dB, 80 dB, or 105 dB.

The methods further comprising repeating delivery of the acoustic stimulus, and measuring a subsequent startle response in the subject. Measuring the initial and subsequent startle response may comprise obtaining the same measurement in the subject. The measurement may be the blink reflex, pupil dilation, skin conductive response, or brain activity in fMRI.

In some embodiments, the method comprises comparing the startle responses (e.g. comparing the initial startle response to the subsequent startle response). Delivery of the acoustic stimulus and measurement of a startle response may be repeated until the subsequent startle response is reduced compared to the initial startle response.

In some embodiments, the acoustic stimulus may comprise a pre-pulse stimulus and a pulse stimulus as described herein. The pre-pulse stimulus may be delivered to the subject prior to the first pulse stimulus. In some embodiments, the pre-pulse stimulus may be delivered to the subject prior to each pulse stimulus. Suitable pre-pulse stimuli and intervals between the pre-pulse stimulus and the pulse stimulus are described above. For example, the pre-pulse stimulus may have an amplitude of 10-100 dB and a frequency of 200-5000 Hz. The pre-pulse stimulus may be provided to the subject 1 ms-10 seconds prior to the pulse stimulus.

In some embodiments, methods for acoustic neuromodulation comprise providing to the subject the acoustic stimulus over multiple treatment sessions, such that the ASR in the subject diminishes over time. The ASR may be measured during the treatment session(s) by any suitable method as described herein. The term “treatment session” as used in the context of acoustic neuromodulation herein refers to the repeated delivery of the acoustic stimulus (e.g. the second delivery, the third delivery, the fourth delivery, etc.) and to the subject and measurement of the subsequent startle response. For example, a treatment session may comprise delivery of an acoustic stimulus (e.g. a pulse stimulus) and measurement of a startle response selected from the blink reflex, pupil dilation, skin conductive response, or brain activity in fMRI. In some embodiments, a first “treatment session” may comprise the initial evaluation of the initial startle response in the subject (e.g. delivery of the acoustic stimulus and measurement of the initial startle response). The treatment phase of the session subsequently comprises the repeated delivery of an acoustic stimulus, and measurement of a subsequent startle response. The second treatment session (which may be performed on the same day or on a different day) may comprise delivery of the acoustic stimulus and measurement of the subsequent startle response. The startle response on the second treatment session (or any subsequent session) may be compared to the initial startle response (e.g. the initial startle response to the first pulse stimulus on the first day of treatment).

Any suitable number of treatment sessions may be provided to the subject to achieve the desired diminishment of ASR in the subject. The subject may receive one treatment session or multiple treatment sessions per day. Treatment sessions may be spaced by any suitable interval. For example, the subject may receive treatment sessions daily, every other day, every three days, every four days, every five days, every six days, every week, every two weeks, every three weeks, monthly, etc. The subject may receive treatment sessions for any suitable duration to achieve the desired result.

In some embodiments, one or multiple startle responses may be measured to evaluate the efficacy of acoustic neuromodulation in the subject. Suitable startle responses include those described above (e.g., fMRI, blink reflex, pupil dilation, skin conductance). In some embodiments, fMRI may be used to evaluate activity in one or more areas of the brain. For example, initial fMRI measurements may be obtained to determine whether a subject has an elevated ASR, as indicated by hyperactivity in one or more areas of the brain. The subject may be exposed to one or more acoustic neuromodulation treatment sessions, during which fMRI may be performed to evaluate whether activity in the one or more areas of the brain diminishes over the course of repeated exposure to the acoustic stimuli. As another example, blink reflex measurements may be used to determine efficacy of acoustic neuromodulation in the subject. For example, the initial blink reflex measurements may be measured to determine whether a subject has an elevated ASR, as indicated by a hyperactive blink reflex. The subject may be exposed to one or more acoustic neuromodulation treatment sessions, during which the blink reflex may be measured to evaluate whether the ASR in the subject diminishes over the course of repeated exposure to the acoustic stimuli.

In some embodiments, acoustic neuromodulation may be performed on a subject diagnosed with or suspected of having a mental disorder. For example, an evaluation of whether a subject has a mental disorder may be performed by a suitable method described herein. As another example, information about whether a subject has a mental disorder may be obtained from a third party, such as a physician. Subjects diagnosed with or suspected of having a mental disorder based upon an abnormal ASR or abnormal PPI may be exposed to acoustic neuromodulation by methods as described herein. Accordingly, methods for acoustic neuromodulation as described herein may be performed to treat a mental disorder in a subject. For example, acoustic neuromodulation may be performed to reduce ASR or improve PPI in a subject with a mental disorder.

### f. Devices

In some aspects, provided herein are devices for delivering a set of acoustic signals as described herein to a subject. The device may comprise a means for delivering the acoustic signal to the subject (e.g., a speaker, headphones, and the like). The device further comprises a control system (e.g., controller/processor) that directs delivery of the acoustic signals to the subject. For example, the control system may comprise a memory component storing software that coordinates the timing and intensity of the signals to be delivered to the subject. The device may further comprise a means for storing data collected from the patient. For example, the device may comprise a memory component for storing fMRI data, skin conductance data, eyeblink data, and/or pupil dilation data collected from the subject.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. National Institute of Health. (2020). Statistics. National Institute of Mental Health. https://www.nimh.nih.gov/health/statistics/index.shtml
2. Dennis, P. A., Kimbrel, A. N., Sherwood, A., Calhoun, P. S., Watkins, L. L., Dennis, M. F., Beckham, J. C. (2017). Trauma and Autonomic Dysregulation: Episodic—Versus Systemic—Negative Affect Underlying Cardiovascular Risk in Posttraumatic Stress Disorder. Psychosomatic Medicine. 79, 5. PMID: 28570433
4. Grillon, C., Morgan, C. A., Southwich, S. M., Davis, M., Charney, D. S. (1995). Baseline startle amplitude and prepulse inhibition in Vietnam veterans with posttraumatic stress disorder. Psychiatry Research. 64. 169-178. PMID: 8944395
5. Jovanovic, T., Blanding, N. Q., Norrholm, S. D., Duncan, E., Bradley, B., Ressler, K. J. (2010). Childhood Abuse is Associated with Increased Startle Reactivity in Adulthood. Depression and Anxiety. 26, 11. 1018-1026. PMID: 19691032
6. Compean, E., Hamner, M. (2019). Posttraumatic stress disorder with secondary psychotic features (PTSD SP): Diagnostic and treatment challenges. Progress in neuro psychopharmacology & biological psychiatry. 88. 265-275. PMID: 30092241
7. Lang, P. J., Bradley, M. M., Cuthbert, B. N. (1990). Emotion, attention, and the startle reflex. Psychology Review. 3. 377-95. PMID: 2200076
8. Lyketsos, G. C. (2007). Psychiatric manifestations of neurologic disease: where are we headed? Dialogs of Clinical Neuroscience. 9, 2. 111-124. PMID: 17726911
9. Valsamis, B., Schmid, S. (2011). Habituation and prepulse inhibition of acoustic startle in rodents. Journal of visualized experiments: JoVE. doi: 10.3791/3446
10. Caine, S. B., Geyer, M. A., Swerdlow, N. R. (1992). Hippocampal modulation of acoustic startle and prepulse inhibition in the rat. Pharmacolofy Biochemistry and Behavior. 43, 4. 1201-1208. https://doi.org/10.1016/0091-3057(92)90503-8

What is claimed is:

1. A set of acoustic signals for delivery to a subject, wherein the set comprises at least one pre-pulse stimulus and at least one pulse stimulus.

2. The set of claim 1, wherein the at least one pre-pulse stimulus has a frequency of 200 Hz, 300 Hz, 400 Hz, 500 Hz, 1000 Hz, 3000 Hz, 4000 Hz, or 5000 Hz and an amplitude of 15 dB, 25 dB, or 45 dB.

3. The set of claim 2, wherein the at least one pre-pulse stimulus has a frequency of 3000 Hz.

4. The set of any of the preceding claims, wherein the at least one pulse stimulus has a frequency of 200 Hz, 300 Hz, 400 Hz, 500 Hz, 1000 Hz, 3000 Hz, 4000 Hz, or 5000 Hz and an amplitude of 65 dB, 80 dB, or 105 dB.

5. The set of claim 4, wherein the at least one pulse stimulus has a frequency of 3000 Hz.

6. A method for evaluating an acoustic startle reflex in a subject, the method comprising:
a. delivering the set of any of the preceding claims to the subject; and
b. measuring a startle response in the subject.

7. The method of claim 6, wherein the startle response is selected from the blink reflex, pupil dilation, skin conductive response, and brain activity in fMRI.

8. The method of claim 7, wherein measuring the blink reflex comprises measuring the speed, magnitude, and/or duration of the blink reflex in the subject.

9. The method of any one of claims 6-8, wherein an increased startle response in the subject indicates that the subject has a mental disorder.

10. The method of claim 7, wherein elevated activity in the hippocampus as measured by fMRI indicates that the subject has post-traumatic stress disorder (PTSD).

11. A method for performing acoustic neuromodulation in a subject, comprising:
a. Delivering an acoustic stimulus to the subject;
b. Measuring an initial startle response in the subject;
c. Delivering the acoustic stimulus to the subject;
d. Measuring a subsequent startle response in the subject;
e. Comparing the startle responses in the subject; and
f. Repeating steps a-d when the subsequent startle response in the subject is not reduced compared to the initial startle response, or ceasing delivery of the acoustic stimulus to the subject when the subsequent startle response in the subject is reduced compared to the initial startle response.

12. The method of claim 11, wherein the acoustic stimulus comprises a pulse stimulus having a frequency of 200 Hz, 300 Hz, 400 Hz, 500 Hz, 1000 Hz, 3000 Hz, 4000 Hz, or 5000 Hz and an amplitude of 65 dB, 80 dB, or 105 dB.

13. The method of claim 12, further comprising delivering a pre-pulse stimulus to the subject prior to each pulse stimulus, wherein the pre-pulse stimulus has a frequency of 200 Hz, 300 Hz, 400 Hz, 500 Hz, 1000 Hz, 3000 Hz, 4000 Hz, or 5000 Hz and an amplitude of 15 dB, 25 dB, or 45 dB.

14. The method of any one of claims 11-13, wherein measuring the initial and subsequent startle response in the subject comprises obtaining the same measurement from the subject.

15. The method of claim 14, wherein the measurement is selected from the blink reflex, pupil dilation, skin conductive response, and brain activity in fMRI.

16. The method of claim 15, wherein measuring the blink reflex comprises measuring the speed, magnitude, and/or duration of the blink reflex in the subject.

17. The method of claim 15, wherein measuring brain activity in fMRI comprises measuring activity in the hippocampus.

18. A method of evaluating paired-pulse inhibition (PPI) in a subject, comprising performing the following steps in order:
b. Delivering a pulse stimulus to the subject;
c. Measuring a first startle response in the subject;
d. Delivering a pre-pulse stimulus to the subject;
e. Delivering the pulse stimulus to the subject;
f. Measuring a second startle response in the subject; and
g. Comparing the second startle response to the first startle response in the subject.

19. The method of claim 18, wherein measuring the first and second startle response comprises obtaining the same measurement from the subject.

20. The method of claim 19, wherein the measurement is selected from the blink reflex, pupil dilation, skin conductive response, and brain activity in fMRI.

21. The method of any one of claims 18-20, wherein the pre-pulse stimulus has a frequency of 200 Hz, 300 Hz, 400 Hz, 500 Hz, 1000 Hz, 3000 Hz, 4000 Hz, or 5000 Hz and an amplitude of 15 dB, 25 dB, or 45 dB.

22. The method of any one of claims 18-21, wherein the pulse stimulus has a frequency of 200 Hz, 300 Hz, 400 Hz, 500 Hz, 1000 Hz, 3000 Hz, 4000 Hz, or 5000 Hz and an amplitude of 65 dB, 80 dB, or 105 dB.
